(19) United States
(12) Patent Application Publication
(10) Pub. No.: US 2022/0307023 A1
(43) Pub. Date: Sep. 29, 2022

(54) ANTISENSE RNA EDITING OLIGONUCLEOTIDES COMPRISING CYTIDINE ANALOGS

(71) Applicants: ProQR Therapeutics II B.V., Leiden (NL); The Regents of the University of California, Oakland, CA (US)

(72) Inventors: Janne Juha Turunen, Leiden (NL); Lenka Van Sint Fiet, Leiden (NL); Cherie Paige Kemmel, Leiden (NL); Peter Beal, Oakland, CA (US); Erin E. Doherty, Oakland, CA (US)

(21) Appl. No.: 17/618,313

(22) PCT Filed: Jun. 12, 2020
(86) PCT No.: PCT/US2020/037580

§ 371 Date: Dec. 10, 2021

Related U.S. Application Data
(60) Provisional application No. 62/860,843, filed on Jun. 13, 2019.

(51) Int. Cl. C12N 15/113; A61K 31/7088

(52) U.S. Cl. CPC C12N 15/113; A61K 31/7088; C12N 2310/11; C12N 2310/315; C12N 2310/321

(57) ABSTRACT

The invention relates to single-stranded RNA editing antisense oligonucleotides (AO Ns) for binding to a target RNA molecule for deaminating at least one target adenosine present in the target RNA molecule and recruiting, in a cell, preferably a human cell, an ADAR2 enzyme, to deaminate the at least one target adenosine in the target RNA molecule. The AON according to the invention comprises a cytidine analog at the position opposite the target adenosine, wherein the cytidine analog serves as an H-bond donor at the N3 site, for more efficient RNA editing.

### CROSS-REFERENCE TO RELATED APPLICATIONS

This application claims priority to U.S. Provisional Application No. 62/860,843, filed Jun. 13, 2019, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 13, 2019, is named 0032WO01ORD_SeqList_ST25.txt and is 2,931 bytes in size.

### TECHNICAL FIELD

The invention relates to the field of medicine. In particular, it relates to the field of RNA editing, whereby an RNA molecule in a cell is targeted by a single stranded antisense oligonucleotide (AON) to specifically change a target nucleotide present in the target RNA molecule. More specifically, the invention relates to RNA-editing AONs that comprise modified nucleotides to improve their in vivo and in vitro RNA editing effect.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, more or less predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result thereof, exons may be included or skipped. The enzymes catalysing adenosine deamination are within an enzyme family of ADARs, which include human deaminases hADAR1 and hADAR2, as well as hADAR3. However, for hADAR3 no deaminase activity has been shown yet.

The use of oligonucleotides to edit a target RNA applying adenosine deaminase has been described (e.g. Woolf et al. 1995. PNAS 92:8298-8302; Montiel-Gonzalez et al. PNAS 2013, 110(45):18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. Clearly, these systems are not readily adaptable for use in humans (e.g. in a therapeutic setting). The oligonucleotides of Woolf et al. (1995) that were 100% complementary to the target RNA sequences, only appeared to function in cell extracts or in Xenopus oocytes by microinjection, and suffered from severe lack of specificity: nearly all adenosines in the target RNA strand that was complementary to the antisense oligonucleotide were edited. An oligonucleotide, 34 nucleotides in length, wherein each nucleotide carried a 2′-O-methyl (2′-OMe) modification, was tested and shown to be inactive in Woolf et al. (1995). To provide stability against nucleases, a 34-mer RNA, modified with 2′-OMe and modified with phosphorothioate (PS) linkages at the 5′- and 3′-terminal 5 nucleotides, was also tested. It was shown that the central unmodified region of this oligonucleotide could promote editing of the target RNA by endogenous ADAR, with the terminal modifications providing protection against exonuclease degradation. However, this system did not show deamination of a specific target adenosine in the target RNA sequence. As mentioned, nearly all adenosines opposite an unmodified nucleotide in the antisense oligonucleotide were edited (therefore nearly all adenosines opposite nucleotides in the central unmodified region, if the 5′- and 3′-terminal 5 nucleotides of the antisense oligonucleotide were modified, or nearly all adenosines in the target RNA strand if no nucleotides were modified).

It is known in the art that ADAR may act on any dsRNA. Through a process sometimes referred to as ‘promiscuous editing’, the enzyme will edit multiple A's in the dsRNA. Hence, there is a need for methods and means that circumvent such promiscuous editing and only target specific adenosines in a target RNA molecule to become therapeutic applicable. Vogel et al. (2014) showed that such off-target editing can be suppressed by using 2′-OMe-modified nucleotides in the oligonucleotide at positions opposite to adenosines that should not be edited, and used a non-modified nucleotide directly opposite to the specifically targeted adenosine on the target RNA. However, the specific editing effect at the target nucleotide has not been shown to take place without the use of recombinant ADAR enzymes having covalent bonds with the AON.

WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion there is no need for conjugated entities or presence of modified recombinant ADAR enzymes. WO2016/097212 describes the recruitment portion as being a stem-loop structure mimicking either a natural substrate (e.g. the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described in WO2016/097212 is an intramolecular stem-loop structure, formed within the AON itself, and able to attract ADAR.

WO2017/220751 and WO2018/041973 describe AONs that do not comprise such a stem-loop structure but that are (almost fully) complementary to the targeted area, except for one or more mismatching nucleotides, or so-called ‘wobbles’, or ‘bulges’. The sole mismatch may be at the site of the nucleotide opposite the target adenosine, but in other embodiments AONs were described with multiple bulges and/or wobbles when attached to the target sequence area. It appeared possible to achieve in vitro, ex vivo and in vivo RNA editing with AONs lacking a stem-loop structure and with endogenous ADAR enzymes when the sequence of the AON was carefully selected such that it could attract ADAR. The ‘orphan nucleotide’, which is defined as the nucleotide in the AON that is positioned directly opposite the target adenosine in the target RNA molecule, did not carry a 2′-OMe modification. The orphan nucleotide could also be a DNA nucleotide (carrying no 2′ modification is the sugar entity), wherein the remainder of the AON did carry 2′-O-alkyl modifications at the sugar entity (such as 2′-OMe), or the nucleotides directly surrounding the orphan nucleotide contained particular chemical modifications (or were DNA) that further improved the RNA editing efficiency and/or increased the resistance against nucleases. Such effects could even be further improved by using sense oligonucleotides (SONs) that ‘protected’ the AONs against breakdown (described in WO2018/134301).

Despite the achievements outlined above, there remains a need for improved compounds that can utilise (endogenous) cellular pathways and enzymes that have deaminase activity, such as naturally expressed ADAR enzymes to more specifically and more efficiently edit endogenous nucleic acids in mammalian cells, even in whole organisms, to alleviate disease.

### SUMMARY OF THE INVENTION

The invention relates to an antisense oligonucleotide (AON) capable of forming a double stranded nucleic acid complex with a target RNA molecule, wherein the double stranded nucleic acid complex is capable of recruiting an ADAR enzyme for deamination of at least one target adenosine in the target RNA molecule, wherein the nucleotide in the AON that is directly opposite the target adenosine is a cytidine analog that serves as an H-bond donor at the N3 site. Preferred cytidine analogs that are used in AONs of the present invention are pseudoisocytidine (piC) and Benner's base Z (dZ). Other preferred cytidine analogs that can be used according to the teaching disclosed herein are 5-hydroxyC-H+, 5-aminoC-H+ and 8-oxoA (syn). Preferably, the cytidine analog does not carry a 2′-OMe or 2′-MOE ribose modification. In a preferred aspect, the AON of the present invention further comprises one or more nucleotides comprising a substitution at the 2′ position of the ribose, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; —O—, S—, or N-allyl; —O-alkyl-O-alkyl; -methoxy; -aminopropoxy; -methoxyethoxy; -dimethylamino oxyethoxy; and -dimethylaminoethoxyethoxy. In another preferred aspect, the ADAR enzyme that is recruited is an endogenous enzyme, preferably an endogenous ADAR2 enzyme.

In another embodiment, the invention relates to a pharmaceutical composition comprising an AON according to the invention and a pharmaceutically acceptable carrier or diluent.

In yet another embodiment, the invention relates to an AON according to the invention, or a pharmaceutical composition according to the invention, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome (such as Usher syndrome type I, type II, and type III), X-linked immunodeficiency, Sturge-Weber Syndrome, and cancer.

In another embodiment, the invention relates to a method for the deamination of a target adenosine present in a target RNA molecule in a cell, the method comprising the steps of: providing the cell with an AON according to the invention, or a pharmaceutical composition according to the invention; allowing annealing of the AON to the target RNA molecule to form a double stranded nucleic acid complex capable of recruiting an endogenous ADAR enzyme in the cell; allowing the ADAR enzyme to deaminate the target adenosine in the target RNA molecule; and optionally identifying the presence of the deaminated adenosine in the target RNA molecule. In yet another embodiment, the invention relates to a method for the deamination of at least one target adenosine present in a target RNA molecule, the method comprising the steps of: providing an AON according to the invention; allowing annealing of the AON to the target RNA molecule to form a double stranded nucleic acid complex; allowing a mammalian ADAR enzyme to deaminate the target adenosine in the target RNA molecule; and optionally identifying the presence of the deaminated adenosine in the target RNA molecule.

### BRIEF DESCRIPTION OF THE DRAWINGS

One or more embodiments of the invention will now be described, by way of example only, with reference to the accompanying drawings, in which:

FIG. 1 shows the crystal structure of the ADAR2 E488Q mutant bound to dsRNA and the contact between the glutamine (Gln) at position 488 and the orphan cytidine.

FIG. 2 shows the protonation-dependent contact between the wild type ADAR2, having a glutamate (Glu) at position 488, and the orphan cytidine.

FIG. 3 shows (on the left) the interaction between the wild type ADAR2, having a glutamate (Glu) at position 488, and the cytidine analog ‘pseudoisocytidine’ (piC) that provides the hydrogen bond donation at N3 to interact with the glutamate residue. On the right the structure of the cytidine analogs piC and Benner's base Z (dZ) are shown, indicating the presence of the hydrogen at N3 (that is absent in normal cytidine).

FIG. 4 shows the two cytidine analogs 5-hydroxycytidine-H+ (left) and 5-aminocytidine-H+ (middle) and an adenosine analog 8-oxoadenosine (right), mimicking a cytidine analog as outlined herein.

FIG. 5 shows the target mouse Idua RNA sequence (5′ to 3′; SEQ ID NO:2) with the target adenosine slightly upwards. Below the target sequence, the 29 nt guide RNA antisense oligonucleotide (from 3′ to 5′; SEQ ID NO:1) is given. N represents an orphan nucleotide (opposite the target adenosine), which may be a normal (deoxy-)cytidine, or a cytidine analog as outlined herein, such as piC or dZ.

FIG. 6 shows the result of the kinetic analysis comparing an AON carrying a normal cytidine (‘C’) opposite the target adenosine and an AON carrying a pseudoisocytidine (piC) opposite the target adenosine.

FIG. 7 shows the results of a kinetic analysis comparing an AON carrying a normal cytidine (deoxy-C, or here: ‘do’) opposite the target adenosine and an AON carrying a Benner's base Z (dZ) opposite the target adenosine.

FIG. 8 shows the results of a kinetic analysis comparing three AONs: one carrying a normal cytidine (deoxy-C, or here: ‘do’) opposite the target adenosine, one AON carrying a Benner's base Z (dZ) as a cytidine analog opposite the target adenosine, and one carrying a deoxy-pseudoisocytidine (dpiC) as a cytidine analog opposite the target adenosine.

FIG. 9 shows (A) the mouse mRNA Idua sequence (lower strand; 5′ to 3′; SEQ ID NO:6) that undergoes the AON targeted A-to-I editing, with the to-be-edited A nucleotide in bold. The upper strand shows from 3′ to 5′ the RNA editing AON sequence (SEQ ID NO:7). The C nucleotide opposing the to-be-edited A is in bold and underlined. (B) shows the two AONs that were tested, here from 5′ to 3′ (same sequence as in (A)). The lower-case nucleotides are 2′-OMe modified RNA nucleotides. The upper-case nucleotides are DNA nucleotides. The asterisks (*) indicate phosphorothioate (PS) linkages. The normal deoxy-cytidine (dC) nucleotide in IDUA287 and the cytidine analog Benner's base Z (dZ) nucleotide in IDUA294 are given in bold and underlined.

FIG. 10 shows the results of a ddPCR analysis of an editing percentage using AONs IDUA287 and IDUA294 after transfection (applying endogenous ADAR), in a primary mouse liver fibroblast cell assay.

### DETAILED DESCRIPTION

There is a constant need for improving the pharmacokinetic properties of RNA-editing antisense oligonucleotides (AONs, sometimes referred to as ‘editing oligonucleotides’, or ‘EONs’) without negatively affecting editing efficiency of the target adenosine in the target RNA. Many chemical modifications exist in the generation of AONs, whose properties are not always compatible with the desire of achieving efficient RNA editing. In the search for better pharmacokinetic properties, it was found earlier that a 2′-O-methoxyethyl (or 2′-methoxyethoxy; or 2′-MOE) modification of the ribose of some, but not all, nucleotides surprisingly appeared compatible with efficient ADAR engagement and editing (WO2019/1548475).

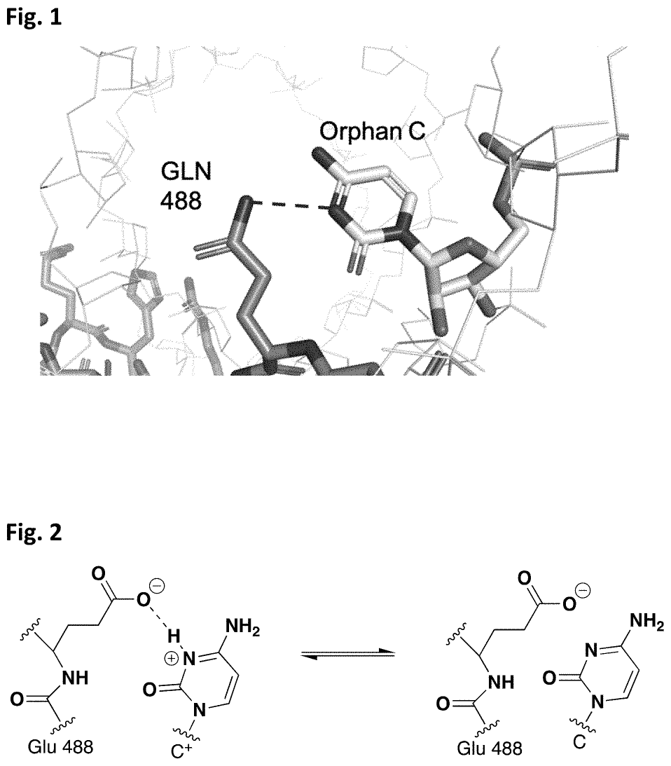

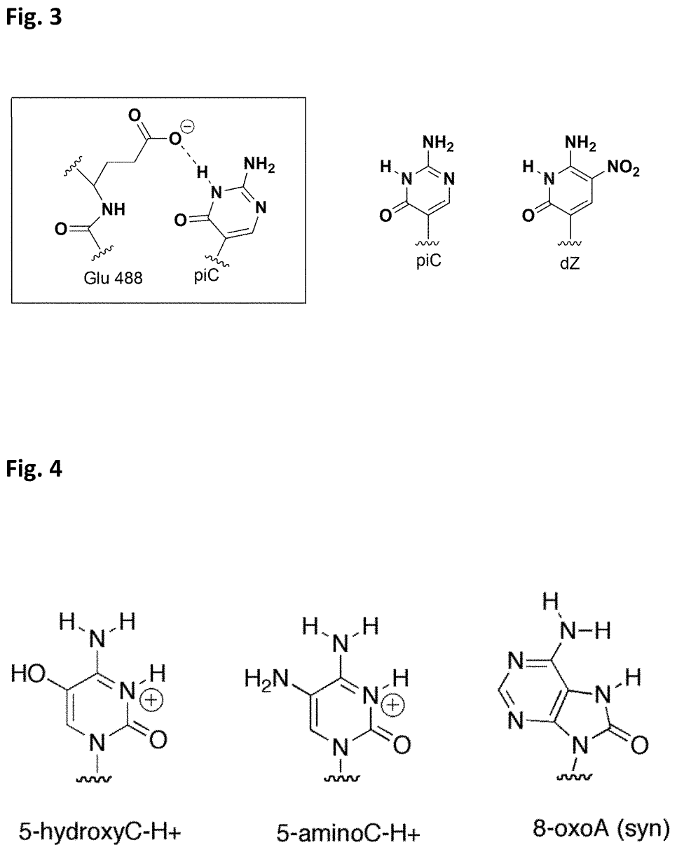

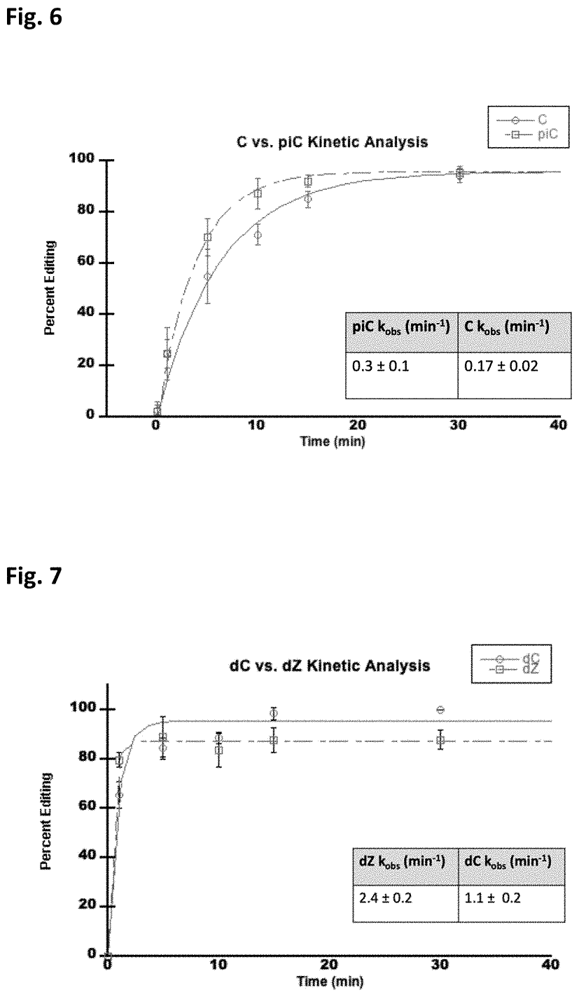

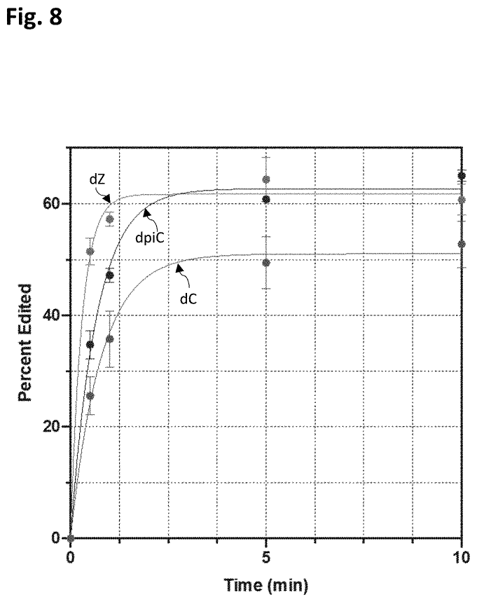

Mutagenesis studies of human ADAR2 revealed that a single mutation at residue 488 from glutamate to glutamine (E488Q), gave an increase in the rate constant of deamination by 60-fold when compared to the wild type enzyme (Kuttan and Bass. Proc Natl Acad Sci USA 2012. 109(48):E3295-3304). During the deamination reaction, ADAR flips the edited base out of its RNA duplex, and into the enzyme active site (Matthews et al. Nat Struct Mol Biol 2016. 23(5):426-433). When ADAR2 edits adenosines in the preferred context (an A:C mismatch) the nucleotide opposite the target adenosine is often referred to as the ‘orphan cytidine’. The crystal structure of ADAR2 E488Q bound to double stranded RNA (dsRNA) revealed that the glutamine (Gin) side chain at position 488 is able to donate an H-bond to the N3 position of the orphan cytidine (FIG. 1) which leads to the increased catalytic rate of ADAR2 E488Q (Kuttan and Bass. 2012). In the wild type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild type situation require protonation for this contact to occur (FIG. 2). In order to make use of endogenously expressed ADAR2 to correct disease relevant mutations (and not mutant ADAR2 versions that may require over-expression and exogenous administration), it is essential to maximize the editing efficiency of the wild type ADAR2 enzyme present in the cell. Instead of using enzyme mutants, the inventors of the present invention aimed to use AONs with modified RNA bases, especially at the position of the orphan cytidine to mimic the hydrogen-bonding pattern observed by the E488Q ADAR2 mutant. By replacing the nucleotide opposite the target adenosine in the AON with particular cytidine analogs that would serve as H-bond donors at N3, it was envisioned that it would be possible to stabilize the same contact that is believed to provide the increase in catalytic rate for the mutant enzyme. FIG. 3 shows two cytidine analogs: pseudoisocytidine (also referred to as ‘piC’; Lu et al. J Org Chem 2009. 74(21):8021-8030; Burchenal et al. (1976) Cancer Res 36:1520-1523) and Benner's base Z (also referred to as ‘dZ’; Yang et al. Nucl Acid Res 2006. 34(21):6095-6101) that were initially selected because they offer hydrogen-bond donation at N3 with minimal perturbation to the shape of the nucleobase. FIG. 4 shows additional non-limiting nucleobase analogs that can be applied for the same purpose. The accompanying examples show a kinetic analysis of ADAR2 that was performed after applying an AON with piC as the nucleotide opposite the target adenosine, in comparison to an AON carrying a normal cytidine at that position. These experiments revealed that the deamination rate when using the piC orphan nucleoside was approximately 1.8 times higher than when an AON was used carrying a normal cytidine as the same position (FIGS. 6 and 8). It was also found that when an AON carrying a normal cytidine (deoxy-C, or dC) opposite the target adenosine was compared to an AON carrying a Benner's Base Z analog (dZ) opposite the target adenosine in a same setup, RNA editing was also improved (FIGS. 7 and 8). Using a transfection setup in primary mouse fibroblasts, RNA editing was also increased when deoxy-C was compared to dZ in a further identical surrounding of the AON (FIG. 10). These results show that the inventors were indeed able to increase deamination efficiency by using an AON carrying a nucleoside analog at the orphan nucleoside position, wherein the N3 site of the analog serves as an H-bond donor site.

The presence of the cytidine analog in the AON of the present invention may also exist in addition to modifications to the ribose 2′ group. The ribose 2′ groups in the AON can be independently selected from 2′-H (i.e. DNA), 2′-OH (i.e. RNA), 2′-OMe, 2′-MOE, 2′-F, or 2′-4′-linked (i.e. a locked nucleic acid or LNA), or other 2′ substitutions. Different 2′ modifications are discussed in further detail in WO2016/097212, WO2017/220751, WO2018/041973, and WO2018/134301. The 2′-4′ linkage can be selected from linkers known in the art, such as a methylene linker or constrained ethyl linker. In all cases, the modifications should be compatible with editing such that the oligonucleotide fulfils its role as an RNA editing AON. The AON may be further optimized for binding to an enzyme with nucleotide deamination activity by generating at least one unlocked nucleic acid (UNA) ribose modification in a position which is not incompatible with editing activity of the enzyme having nucleotide deaminase activity (as described in PCT/EP2020/053283, unpublished). In a UNA modification, there is no carbon-carbon bond between the ribose 2′ and 3′ carbon atoms. UNA ribose modifications therefore increase the local flexibility in oligonucleotides. UNAs can lead to effects such as improved pharmacokinetic properties through improved resistance to degradation. UNAs can also decrease toxicity and may participate in reducing off-target effects. Preferably, the AON is an RNA editing single-stranded AON that targets a pre-mRNA or an mRNA, wherein the target nucleotide is an adenosine in the target RNA, wherein the adenosine is deaminated to an inosine, which is being read as a guanosine by the translation machinery. Preferably, the adenosine is located in a UGA or UAG stop codon, which is edited to a UGG codon; or wherein two target nucleotides are the two adenosines in a UAA stop codon, which codon is edited to a UGG codon through the deamination of both target adenosines, wherein two nucleotides in the oligonucleotide mismatch with the target nucleic acid.

The AON according to the invention can comprise internucleoside linkage modifications. In one embodiment one such other internucleoside linkage can be a phosphonoacetate, phosphorothioate (PS) or a methylphosphonate (MP) modified linkage. A preferred linkage is a PS linkage. Preferred positions for MP linkages are described in PCT/EP2020/059369 (unpublished). In another embodiment, the internucleotide linkage can be a phosphodiester wherein the OH group of the phosphodiester has been replaced by alkyl, alkoxy, aryl, alkylthio, acyl, —NR1R1, alkenyloxy, alkynyloxy, alkenylthio, alkynylthio, —S-Z+, —Se-Z+, or —BH3-Z+, and wherein R1 is independently hydrogen, alkyl, alkenyl, alkynyl, or aryl, and wherein Z+ is ammonium ion, alkylammonium ion, heteroaromatic iminium ion, or heterocyclic iminium ion, any of which is primary, secondary, tertiary or quaternary, or Z is a monovalent metal ion, and is preferably a PS linkage.

In the AON of the present invention, the orphan nucleotide (the nucleotide directly opposite the target adenosine) generally comprises a ribose with a 2′—OH group, or a deoxyribose with a 2′-H group, and preferably does not comprise a ribose carrying a 2′-OMe modification. Further, the AON of the present invention generally does not comprise 2′-MOE modifications at certain positions relative to the orphan nucleotide, and further may comprise 2′-MOE modifications at other positions within the AON.

The invention relates to a method for the deamination of at least one target adenosine present in a target RNA molecule in a cell, the method comprising the steps of providing the cell with an AON according to a first aspect of the invention, or a composition according to a second aspect of the invention, allowing uptake by the cell of the AON, allowing annealing of the AON to the target RNA molecule, allowing a mammalian enzyme with nucleotide deaminase activity to deaminate the target nucleotide in the target RNA molecule, and optionally identifying the presence of the deaminated nucleotide in the target RNA molecule. Preferably, the presence of the target RNA molecule is detected by either (i) sequencing the target sequence, (ii) assessing the presence of a functional, elongated, full length and/or wild type protein when the target adenosine is located in a UGA or UAG stop codon, which is edited to a UGG codon through the deamination, (iii) assessing the presence of a functional, elongated, full length and/or wild type protein when two target adenosines are located in a UAA stop codon, which is edited to a UGG codon through the deamination of both target adenosines, (iv) assessing whether splicing of the pre-mRNA was altered by the deamination; or (v) using a functional read-out, wherein the target RNA after the deamination encodes a functional, full length, elongated and/or wild type protein. The present invention therefore also relates to AONs that target premature termination stop codons (PTCs) present in the (pre)mRNA to alter the adenosine present in the stop codon to an inosine (read as a G), which in turn then results in read-through during translation and a full length functional protein. The teaching of the present invention, as outlined herein, is applicable for all genetic diseases that may be targeted with AONs and may be treated through RNA editing.

In a preferred embodiment, the AON according to the invention comprises 2, 3, 4, 5, 6, 7, 8, 9 or 10 mismatches, wobbles and/or bulges with the complementary target RNA region. When the nucleotide opposite the target adenosine is a cytidine analog, the AON mismatches at least once with the target RNA molecule. However, in a preferred aspect one or more additional mismatching nucleotides, wobbles and/or bulges are present between AON and target RNA. These should add to the RNA editing efficiency by the ADAR present in the cell, at the target adenosine position. The person skilled in the art can determine whether hybridization under physiological conditions still does take place. The AON of the present invention can recruit (engage) a mammalian ADAR enzyme present in the cell, wherein the ADAR enzyme comprises its natural dsRNA binding domain as found in the wild type enzyme. The AONs according to the present invention can utilise endogenous cellular pathways and naturally available ADAR enzyme, or enzymes with ADAR activity (which may be yet unidentified ADAR-like enzymes) to specifically edit a target adenosine in a target RNA sequence. As disclosed herein, the single-stranded AONs of the invention are capable of deamination of a specific target, such as adenosine, in a target RNA molecule. Ideally, at least one target nucleotide is deaminated. Alternatively, 1, 2, or 3 further nucleotides are deaminated. Taking the features of the AONs of the present invention together, there is no need for modified recombinant ADAR expression, there is no need for conjugated entities attached to the AON, or the presence of long recruitment portions that are not complementary to the target RNA sequence. Besides that, the AON of the present invention does allow for the specific deamination of a target nucleotide present in the target RNA molecule by a natural nucleotide deaminase enzyme comprising a natural dsRNA binding domain as found in the wild type enzyme, without the risk of promiscuous editing elsewhere in the RNA/AON complex.

The invention relates to an AON capable of forming a double stranded nucleic acid complex with a target RNA molecule, wherein the double stranded nucleic acid complex is capable of recruiting an ADAR enzyme for deamination of at least one target adenosine in the target RNA molecule, wherein the nucleotide in the AON that is directly opposite the at least one target adenosine is a cytidine analog that serves as an H-bond donor at the N3 site. Preferably, the cytidine analog is pseudoisocytidine (piC) or Benner's base Z (dZ). These cytidine analog nucleotides can come in an RNA or DNA format, or potentially modified at the 2′ position as outlined further herein. Other preferred cytidine analogs that can be used in the AON of the present invention are 5-hydroxyC-H+, 5-aminoC-H+ and 8-oxoA (syn). Other cytidine analogs that can also be used in oligonucleotides according to the invention are derivatives of pseudoisocytidine (piC), Benner's base Z (dZ), 5-hydroxyC-H+, 5-aminoC-H+ and 8-oxoA (syn), such as cytidine C5 methyl, ethyl, propyl, etc., variants of the Benner' base Z that have different substituents than nitro (e.g. alkyl, F, Cl, Br, CN, etc.) and variants of 8-oxoA that are substituted at C2 (methyl, ethyl, propyl, halogens, etc. In one preferred aspect, the cytidine analog does not carry a 2′-OMe or 2′-MOE ribose modification. In another preferred aspect, the AON according to the present invention comprises at least one phosphorothioate (PS), phosphonoacetate and/or methylphosphonate (MP) internucleotide linkage. In a preferred aspect, the double stranded nucleic acid complex can recruit an endogenous ADAR enzyme, preferably wherein the ADAR enzyme is an endogenous ADAR2 enzyme. In another preferred aspect, the AON comprises at least 15, 16, 17, 18, 19, 20, 21, 22, 23, 24, 25, 26, 27, 28, 29, 30, 31, 32, 33, 34, 35, or 36 nucleotides, and is at most 100 nucleotides long, preferably at most 60 nucleotides long.

The invention also relates to a pharmaceutical composition comprising an AON according to the invention, and a pharmaceutically acceptable carrier or diluent. Such pharmaceutically acceptable carriers or diluents are well known to the person skilled in the art.

In another embodiment, the invention relates to an AON according to the invention, or a pharmaceutical composition according to the invention, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome (such as Usher syndrome type I, type II, and type III), X-linked immunodeficiency, Sturge-Weber Syndrome, and cancer.

In another embodiment, the invention relates to a method for the deamination of a target adenosine present in a target RNA molecule in a cell, the method comprising the steps of: providing the cell with an AON according to the invention, or a pharmaceutical composition according to the invention; allowing annealing of the AON to the target RNA molecule to form a double stranded nucleic acid complex capable of recruiting an endogenous ADAR enzyme in the cell; allowing the ADAR enzyme to deaminate the target adenosine in the target RNA molecule; and optionally identifying the presence of the deaminated adenosine in the target RNA molecule. The optional step in identifying the presence of the deaminated adenosine is performed by: sequencing a region of the target RNA molecule, wherein the region comprises the deaminated target adenosine; assessing the presence of a functional, elongated, full length and/or wild type protein when the target adenosine is located in a UGA or UAG stop codon; assessing the presence of a functional, elongated, full length and/or wild type protein when two target adenosines are located in a UAA stop codon; assessing, when the target RNA molecule is pre-mRNA, whether splicing of the pre-mRNA was altered by the deamination; or using a functional read-out, wherein the target RNA molecule after the deamination encodes a functional, full length, elongated and/or wild type protein.

In yet another embodiment, the invention relates to a method for the deamination of at least one target adenosine present in a target RNA molecule, the method comprising the steps of: providing an AON according to the invention; allowing annealing of the AON to the target RNA molecule to form a double stranded nucleic acid complex; allowing a mammalian ADAR enzyme to deaminate the target adenosine in the target RNA molecule; and optionally identifying the presence of the deaminated adenosine in the target RNA molecule.

The double stranded AON/target RNA molecule complex interacts through Watson-Crick base-pairing. The skilled person is able, based on the teaching available in the art, to determine the level of capability to achieve RNA editing and compare this to an AON lacking specific sugar- and/or linkage modifications specified positions. In another preferred aspect, the AON of the invention further comprises one or more nucleotides comprising a substitution at the 2′ position of the ribose, wherein the substitution is selected from the group consisting of: —OH; —F; -substituted or unsubstituted, linear or branched lower (C1-C10) alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; —O—, S—, or N-allyl; —O-alkyl-O-alkyl; -methoxy; -aminopropoxy; -methoxyethoxy (2′-MOE); -dimethylamino oxyethoxy; and -dimethylaminoethoxyethoxy.

The nucleotide in the AON that is directly opposite the target nucleotide is herein defined as the ‘orphan nucleotide’. In a preferred embodiment, the AON of the present invention comprises at least one nucleotide comprising a 2′-OMe or a 2′-MOE ribose modification, and the orphan nucleotide does not carry a 2′-OMe or a 2′-MOE ribose modification.

The AON according to the present invention is preferably at least 15, 16, 17, 18, 19, 20, 21, 22, 23, 24, 25, 26, 27, 28, 29, 30, 31, 32, 33, 34, 35, or 36 nucleotides in length. The length of the AON may vary depending on the structures that are present (hairpin-structured AONs are generally longer, but when no hairpin structure is present, the AON may be relatively ‘short’, preferably comprising 15 to 25 nucleotides). The AON of the present invention does not necessarily carry a recruiting portion (a stem-loop structure) to attract ADAR, but it is not excluded. In any case, the cytidine analogs as outlined herein may be applied in a variety of different RNA editing AONs. Also, preferably, the AON is shorter than 100 nucleotides, more preferably shorter than 60 nucleotides.

In another embodiment, the invention relates to the use of an AON according to the invention in the manufacture of a medicament for the treatment of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome (such as Usher syndrome type I, type II, and type III), X-linked immunodeficiency, Sturge-Weber Syndrome, and cancer.

The mammalian enzyme with nucleotide deaminase activity that is engaged through the use of the AON according to the invention is preferably an adenosine deaminase enzyme, more preferably ADAR2, even more preferably an endogenous ADAR2 enzyme present in a cell, and is capable of altering the target nucleotide in the target RNA molecule, which target nucleotide is then preferably an adenosine that is deaminated to an inosine.

In another embodiment, the invention relates to a method of treating a subject, preferably a human subject in need thereof, wherein the subject suffers from a genetic disorder caused by a mutation involving the appearance of an adenosine (for instance in a PTC), and in which deamination of the target adenosine to an inosine would alleviate, prevent, or ameliorate the disease, comprising the steps of administering to the subject an AON or pharmaceutical composition according to the invention, allowing the formation of a double stranded nucleic acid complex of the AON with its specific complementary target nucleic acid in a cell in the subject; allowing the engagement of an endogenous present hADAR2; and allowing the enzyme to deaminate the target adenosine in the target nucleic target molecule to an inosine, thereby alleviating, preventing or ameliorating the genetic disease. The genetic diseases that may be treated according to this method are preferably, but not limited to the genetic diseases listed herein (see above).

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar. A base, sometimes called a nucleobase, is generally adenine, cytosine, guanine, thymine or uracil, or a derivative thereof. Cytosine, thymine, and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate (PS), such a moiety is still referred to as the backbone linkage of the monomer. This is referred to as a “backbone linkage modification”. In general terms, the backbone of an oligonucleotide comprises alternating scaffolds and backbone linkages.

The nucleobases in an oligonucleotide of the present invention can be adenine, cytosine, guanine, thymine, or uracil. The nucleobases can be a modified form of adenine, cytosine, guanine, or uracil. The modified nucleobase can be hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-am inomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar.

The oligonucleotide of the present invention may comprise one or more nucleotides carrying a 2′-O-methoxyethyl (2′-MOE) ribose modification. Also, the AON preferably comprises one or more nucleotides not carrying a 2′-MOE ribose modification, and wherein the 2′-MOE ribose modifications are at positions that do not prevent the enzyme with nucleotide deaminase activity from deaminating the target nucleotide. And in another preferred aspect, the AON comprises 2′-O-methyl (2′-OMe) ribose modifications at the positions that do not comprise a 2′-MOE ribose modification, and/or wherein the oligonucleotide comprises deoxynucleotides at positions that do not comprise a 2′-MOE ribose modification. The AON may comprise one or more nucleotides comprising a 2′ position comprising 2′-MOE, 2′-OMe, 2′-OH, 2′-deoxy, 2′-F, or a 2′-4′-linkage (i.e. a locked nucleic acid or LNA). The 2′-4′ linkage can be selected from linkers known in the art, such as a methylene linker or constrained ethyl linker. Different 2′ modifications are discussed in further detail in WO2016/097212, WO2017/220751, WO2018/041973, WO2018/134301, WO2019/219581, and WO2019/158475. In all cases, the modifications should be compatible with editing such that the oligonucleotide fulfils its role as an editing oligonucleotide. Where a position comprises a UNA ribose modification, that position can have a 2′ position comprising the same modifications discussed above, i.e. 2′-MOE, 2′-OMe, 2′-OH, 2′-deoxy, 2′-F, or a 2′-4′-linkage (i.e. a locked nucleic acid or LNA). Again, in all cases, the modifications should be compatible with editing such that the oligonucleotide fulfils its role as an editing oligonucleotide. In all aspects of the invention, the enzyme with nucleotide deaminase activity is preferably ADAR1 or ADAR2. In a highly preferred embodiment, the AON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. In a further preferred embodiment, the adenosine is located in a UGA or UAG stop codon, which is edited to a UGG codon; or wherein two target nucleotides are the two adenosines in a UAA stop codon, which codon is edited to a UGG codon through the deamination of both target adenosines, wherein two nucleotides in the oligonucleotide mismatch with the target nucleic acid. The invention also relates to a pharmaceutical composition comprising the AON as characterized herein, and a pharmaceutically acceptable carrier.

The term ‘cytidine analog’ refers to any nucleobase that serves as an H-bond donor at N3 to interact with ADAR2. Non-limiting examples of such cytidine analogs are pseudoisocytidine (piC), Benner's Z (dZ), 5-hydroxyC-H+, 5-aminoC-H+ and 8-oxoA (syn). The skilled person will be aware, based on the present disclosure, that any nucleobase that serves as an H-bond donor at N3 to interact with hADAR2, and that allow deamination of the target adenosine is within the definition of a cytidine analog as used herein. The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group), an unlocked nucleic acid (UNA), a nucleotide including a linker comprising a phosphodiester, phosphonoacetate, phosphotriester, PS, phosphoro(di)thioate, MP, phosphoramidate linkers, and the like. Sometimes the terms adenosine and adenine, guanosine and guanine, cytidine and cytosine, uracil and uridine, thymine and thymidine/uridine, inosine and hypo-xanthine, are used interchangeably to refer to the corresponding nucleobase on the one hand, and the nucleoside or nucleotide on the other. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated above, a nucleotide is a nucleoside+one or more phosphate groups. The terms ‘ribonucleoside’ and ‘deoxyribonucleoside’, or ‘ribose’ and ‘deoxyribose’ are as used in the art. Whenever reference is made to an ‘antisense oligonucleotide’, ‘oligonucleotide’, or ‘AON’ both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I.

In a preferred aspect, the AON of the present invention is an oligoribonucleotide that comprises chemical modifications and may include deoxynucleotides (DNA) at certain specified positions. Terms such as oligonucleotide, oligo, ON, oligonucleotide composition, antisense oligonucleotide, AON, (RNA) editing oligonucleotide, EON, and RNA (antisense) oligonucleotide may be used herein interchangeably. Whenever reference is made to nucleotides in the oligonucleotide construct, such as cytosine, 5-methylcytosine, 5-hydroxymethylcytosine and β-D-Glucosyl-5-hydroxy-methylcytosine are included; when reference is made to adenine, N6-Methyladenine and 7-methyladenine are included; when reference is made to uracil, dihydrouracil, 4-thiouracil and 5-hydroxymethyluracil are included; when reference is made to guanine, 1-methylguanine is included. Whenever reference is made to nucleosides or nucleotides, ribofuranose derivatives, such as 2′-desoxy, 2′-hydroxy, and 2′-O-substituted variants, such as 2′-O-methyl, are included, as well as other modifications, including 2′-4′ bridged variants. Whenever reference is made to oligonucleotides, linkages between two mononucleotides may be phosphodiester linkages as well as modifications thereof, including, phosphonoacetate, phosphodiester, phosphotriester, PS, phosphoro(di)thioate, MP, phosphor-amidate linkers, and the like.

The term ‘comprising’ encompasses ‘including’ as well as ‘consisting’, e.g. a composition ‘comprising X’ may consist exclusively of X or may include something additional, e.g. X+Y. The term ‘about’ in relation to a numerical value x is optional and means, e.g. x±10%. The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the AON hybridizes under physiological conditions to the target sequence. The term does not mean that each and every nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence such that the cellular RNA editing enzymes can edit the target adenosine. The term “substantially complementary” therefore also means that in spite of the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, if under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. Mismatched nucleotides are G-A, C-A, U-C, A-A, G-G, C-C, U-U pairs. In some embodiments AONs of the present invention comprise fewer than four mismatches, for example 0, 1 or 2 mismatches. Wobble base pairs are: G-U, I-U, I-A, and I-C base pairs.

The term ‘splice mutation’ relates to a mutation in a gene that encodes for a pre-mRNA, wherein the splicing machinery is dysfunctional in the sense that splicing of introns from exons is disturbed and due to the aberrant splicing the subsequent translation is out of frame resulting in premature termination of the encoded protein. Often such shortened proteins are degraded rapidly and do not have any functional activity, as discussed herein. The exact mutation does not have to be the target for the RNA editing; it may be that a neighbouring or nearby adenosine in the splice mutation is the target nucleotide, which conversion to I fixes the splice mutation back to a normal state. The skilled person is aware of methods to determine whether normal splicing is restored, after RNA editing of the adenosine within the splice mutation site or area.

An AON according to the present invention may be chemically modified almost in its entirety, for example by providing nucleotides with a 2′-O-methylated sugar moiety (2′-OMe) and/or with a 2′-O-methoxyethyl sugar moiety (2′-MOE). However, the orphan nucleotide is a cytidine analog and preferably does not comprise the 2′-OMe or 2′-MOE modification, and in yet a further preferred aspect, at least one and in a preferred aspect both the two neighbouring nucleotides flanking each nucleotide opposing the target adenosine further do not comprise a 2′-OMe modification. Complete modification wherein all nucleotides of the AON hold a 2′-OMe modification results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group, or by providing a guanine or adenine as opposing base, as these two nucleobases are also able to reduce editing of the opposing adenosine. Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In a preferred aspect, the AON of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art, that RNA editing entities (such as human ADAR enzymes) edit dsRNA structures with varying specificity, depending on a number of factors. One important factor is the degree of complementarity of the two strands making up the dsRNA sequence. Perfect complementarity of the two strands usually causes the catalytic domain of hADAR to deaminate adenosines in a non-discriminative manner, reacting more or less with any adenosine it encounters. The specificity of hADAR1 and 2 can be increased by introducing chemical modifications and/or ensuring a number of mismatches in the dsRNA, which presumably help to position the dsRNA binding domains in a way that has not been clearly defined yet. Additionally, the deamination reaction itself can be enhanced by providing an AON that comprises a mismatch opposite the adenosine to be edited. The mismatch as disclosed herein is created by providing a targeting portion having a cytidine analog opposite the adenosine to be edited. Following the instructions in the present application, those of skill in the art will be capable of designing the complementary portion of the oligonucleotide according to their needs.

The RNA editing protein present in the cell that is of most interest to be used with AONs of the present invention is human ADAR2. It will be understood by a person having ordinary skill in the art that the extent to which the editing entities inside the cell are redirected to other target sites may be regulated by varying the affinity of the AONs according to the invention for the recognition domain of the editing molecule. The exact modification may be determined through some trial and error and/or through computational methods based on structural interactions between the AON and the recognition domain of the editing molecule. In addition, or alternatively, the degree of recruiting and redirecting the editing entity resident in the cell may be regulated by the dosing and the dosing regimen of the AON. This is something to be determined by the experimenter (in vitro) or the clinician, usually in phase I and/or II clinical trials.

The invention concerns the modification of target RNA sequences in eukaryotic, preferably metazoan, more preferably mammalian, most preferably human cells. The invention can be used with cells from any organ e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject. The cell can be located in vitro, ex vivo or in vivo. One advantage of the invention is that it can be used with cells in situ in a living organism, but it can also be used with cells in culture. In some embodiments cells are treated ex vivo and are then introduced into a living organism (e.g. re-introduced into an organism from whom they were originally derived). The invention can also be used to edit target RNA sequences in cells within a so-called organoid. Organoids can be thought of as three-dimensional in vitro-derived tissues but are driven using specific conditions to generate individual, isolated tissues (e.g. see Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound by theory, the RNA editing through hADAR2 is thought to take place on primary transcripts in the nucleus, during transcription or splicing, or in the cytoplasm, where e.g. mature mRNA, miRNA or ncRNA can be edited.

Many genetic diseases are caused by G to A mutations, and these are preferred target diseases because adenosine deamination at the mutated target adenosine will reverse the mutation to a codon giving rise to a functional, full length and/or wild type protein, especially when it concerns PTCs. Preferred examples of genetic diseases that can be prevented and/or treated with oligonucleotides according to the invention are any disease where the modification of one or more adenosines in a target RNA will bring about a (potentially) beneficial change. Especially preferred are Usher syndrome and CF, and more specifically the RNA editing of adenosines in the disease-inducing PTCs in CFTR RNA is preferred. Those skilled in the art of CF mutations recognise that between 1000 and 2000 mutations are known in the CFTR gene, including G542X, W1282X, R553X, R1162X, Y122X, W1089X, W846X, W401X, 621+1G>T or 1717-1G>A.

It should be clear, that targeted editing according to the invention can be applied to any adenosine, whether it is a mutated or a wild-type nucleotide in a given sequence. For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether or not to prevent, delay or treat disease or for any other purpose, in medicine or biotechnology, as diagnostic, prophylactic, therapeutic, research tool or otherwise, are encompassed by the present invention.

The amount of AON to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of AON could compete for binding to an ADAR within a cell, thereby depleting the amount of the entity which is free to take part in RNA editing, but routine dosing trials will reveal any such effects for a given AON and a given target.

One suitable trial technique involves delivering the AON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively the change may be assessed on the level of the protein (length, glycosylation, function or the like), or by some functional read-out, such as a(n) (inducible) current, when the protein encoded by the target RNA sequence is an ion channel, for example.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

AONs of the invention are particularly suitable for therapeutic use, and so the invention provides a pharmaceutical composition comprising an AON of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g. syringe, inhaler, nebuliser) which includes a pharmaceutical composition of the invention.

The invention also provides an AON of the invention for use in a method for making a change in a target RNA sequence in a mammalian, preferably a human cell, as described herein. Similarly, the invention provides the use of an AON of the invention in the manufacture of a medicament for making a change in a target RNA sequence in a mammalian, preferably a human cell, as described herein.

The invention also relates to a method for the deamination of at least one specific target adenosine present in a target RNA sequence in a cell, the method comprising the steps of: providing the cell with an AON according to the invention; allowing uptake by the cell of the AON; allowing annealing of the AON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The AON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, a gel, an eye-drop, or in any other form compatible with pharmaceutical use in humans.

### EXAMPLES

### Example 1: Synthesis of RNA-Editing Guide Antisense Oligonucleotides Containing Pseudoisocytidine (piC) at the Position Opposite the Target Adenosine

All reagents were purchased from Combi-blocks, Sigma Aldrich, or Fisher Scientific and were used without further purification unless noted otherwise. Reactions requiring anhydrous conditions were carried out under an atmosphere of dry argon. Liquid reagents were introduced with either plastic disposable syringes or oven-dried glass microsyringes. Pyridine and N,N-diisopropylethylamine were distilled from CaH₂ and stored over activated 3 Å molecular sieves. Tetrahydrofuran and 1-methylimidazole were dried over 3 Å molecular sieves for 16 h. Dichloromethane was used directly from a Pure Process Technology solvent purification system. Starting material for reactions requiring anhydrous conditions was dried by co-evaporating with anhydrous acetonitrile, then 10% (v/v) anhydrous pyridine in dichloromethane. Thin-layer chromatography (TLC) was performed with Merck silica gel 60 F254 precoated TLC plates. Flash column chromatography was performed on Fisher Scientific Grade 60 (230-400 Mesh) silica gel. ¹H, ¹³C, and ³¹P NMR was performed on a Bruker 400 MHz NMR. The intermediates for the piC building block:
i) 6-N-(Dimethylformamidino)pseudoisocytidine;
ii) 5′-O-(4,4-Dimethoxytrityl)-6-N-(dimethylformamidino)pseudoisocytidine;
iii) 2′-O-(tert-Butyl)dimethylsilyl-5′-O-(4,4-dimethoxytrityl)-6N(dimethylformamidino)pseudoisocytidine; and
iv) 3-[2′-O-(tert-Butyl)dimethylsilyl-3′-O-(2-cyanoethyl-N, N-diisopropylphosphino)-5′-O-(4,4-dimethoxytrityl)-β-D-ribofuranosyl]-6-N-(dimethylformamidino)pseudoisocytidine were all synthesized according to literature procedures, using methods known to the person skilled in the art.

The AON containing the piC cytidine analog was generated using methods known to the person skilled in the art. Modifications of the AON are given in the legend of the figures. The AON containing the dZ cytidine analog can be manufactured according to methods known to the person skilled in the art. This AON contained the same modifications as the AON containing the piC.

### Example 2: Kinetic ADAR Assay Comparing a Normal Cytidine (C) and Pseudoisocytidine (piC) as the Orphan Nucleotide in an RNA Editing Antisense Oligonucleotide

Distilled, deionized water was used for all aqueous reactions and dilutions. Benner's base Z was purchased from FireBird Biomolecular Sciences LLC as a deoxyribonucleoside phosphoramidite. Molecular-biology-grade bovine serum albumin (BSA), and RNase inhibitor were purchased from New England BioLabs. SDS-polyacrylamide gels were visualized with a Molecular Dynamics 9400 Typhon phosphorimager. Data were analyzed with Molecular Dynamics ImageQuant 5.2 software. All MALDI analyses were performed at the University of California, Davis Mass Spectrometry Facilities using a Bruker UltraFlextreme MALDI TOF/TOF mass spectrometer. Oligonucleotide masses were determined with Mongo Oligo Calculator v2.08. Unless otherwise noted, unmodified oligonucleotides were purchased from either Dharmacon or Integrated DNA Technologies.

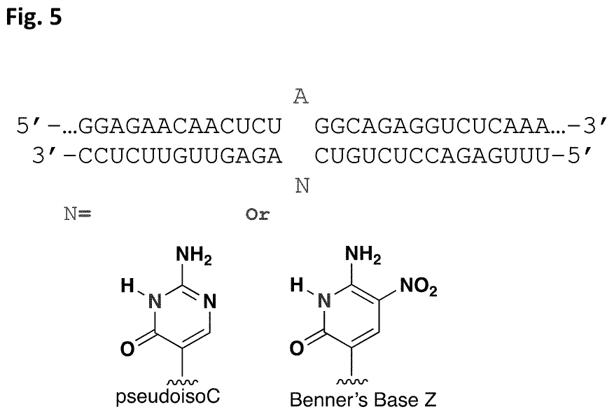

RNA chemical synthesis for cytidine analog-containing AONs was performed at the University of Utah using an ABI 394 synthesizer. Nucleosides were incorporated during the appropriate cycle. Sequence of the AONs was 5′-UUU GAG ACC UCU GUC C*AG AGU UGU UCU CC-3′ (SEQ ID NO:1, with C* being piC or dZ, shown as N in FIG. 5). Single-stranded AONs were purified by denaturing polyacrylamide gel electrophoresis and visualized by UV shadowing. Bands were excised from the gel, crushed and soaked overnight at 4° C. in 0.5M NaOAc, 0.1% sodium dodecyl sulfate (SDS), and 0.1 mM EDTA. Polyacrylamide fragments were removed with a 0.2 μm filter, and the RNAs were precipitated from a solution of 75% EtOH at -70° C. for 4 hrs. The solution was centrifuged 13,000 rpm for 20 min and supernatant was removed. RNA solutions were lyophilized to dryness, resuspended in nuclease-free water, and quantified by absorbance at 260 nm. Oligonucleotide mass was confirmed by MALDI TOF. Purified top and bottom strands were added in a 10:1 ratio to hybridization buffer (180 nM edited strand, 1.8 μM guide strand, 1×TE Buffer, 100 mM NaCl), heated to 95° C. for 5 min, and slowly cooled to room temperature.

Wild type hADAR2 was expressed and as previously described (Matthews et al. 2016; MacBeth and Bass. Methods Enzymol. 2007. 15(424):319-331). Purification of hADAR2 was carried out by lysing cells in buffer containing 20 mM Tris-HCl, pH 8.0, 5% glycerol, 1 mM 2-mercaptoethanol, 750 mM NaCl, 35 mM imidazole, and 0.01% Nonidet P-40 using a French press. Cell lysate was clarified by centrifugation (19,000 rpm for 1 hr). Lysate was passed over a 3 mL Ni-NTA column, which was then washed in 3 steps with 20 mL lysis buffer, wash I buffer (20 nM Tris-HCl, pH 8.0, 5% glycerol, 1 mM 2-mercaptoethanol, 750 mM NaCl, 35 mM imidazole, 0.01% Nonidet P-40), wash II buffer (20 mM Tris-HCl, pH 8.0, 5% glycerol, 1 mM 2-mercaptoethanol, 35 mM imidazole, 500 mM NaCl), and eluted with 20 mM Tris-HCl, pH 8.0, 5% glycerol, 1 mM 2-mercaptoethanol, 400 mM imidazole, 100 mM NaCl. Fractions containing the target protein were pooled and concentrated to 30-80 μM for use in biochemical assays. Protein concentrations were determined using BSA standards visualized by SYPRO orange staining of SDS-polyacrylamide gels. Purified hADAR2 wt was stored in 20 mM Tris-HCl pH 8.0, 100 mM NaCl, 20% glycerol and 1 mM 2-mercaptoethanol at −70° C.

Target RNA was transcribed from a DNA template with the MEGAScript T7 Kit (ThermoFisher). DNA Digestion was performed using RQ1 RNase-free DNase (Promega). DNase treated RNA product was purified as described above.

DNA Template Sequence:

(SEQ ID NO: 3)
TAATACGACTCACTATAGGGctcctcccatcctgtgggctgaacagtat
aacagactcccagtatacaaatggtgggagctagatattagggtaggaa
gccagatgctaggtatgagagagccaacagcctcagccctctgcttggc
ttatagATGGAGAACAACTCT [symbol] GGCAGAGGTCTCAAAGGCTGGGGCTG
TGTTGGACAGCAATCATACAGTGGGTGTCCTGGCCAGCACCCATCACCC
TGAAGGCTCCGCAGCGGCCTGGAGTACCACAGTCCTCATCTACACTAGT
GATGACACCCACGCACACCCCggatcc

With the italic region representing the T7 promoter, with the bold large A representing the target adenosine, with ggatcc being the restriction site (BamHI), and with the underlined region representing the target sequence shown in FIG. 5.

Primers for RT-PCR were ‘Target FWD’: 5′-GCT CCT CCC ATC CTG TGG GCT GAA CAG T-3′ (SEQ ID NO:4) and ‘Target RVS’: 5′-CGG GGT GTG CGT GGG TGT CAT CAC T-3′ (SEQ ID NO:5).

Deamination assays were performed under single-turnover conditions in 15 mM Tris-HCl pH 7.5, 3% glycerol, 60 mM KCl, 1.5 mM EDTA, 0.003% Nonidet P-40, 3 mM MgCl₂, 160 U/mL RNAsin, 1.0 μg/mL, 0.8 nM RNA, and 2 nM ADAR2 wt enzyme. Each reaction solution was incubated at 30° C. for 30 min before adding enzyme and allowed to incubate at 30° C. for varying times prior to stopping with 190 μL 95° C. water and heating at 95′C for 5 minutes. RT-PCR (Promega Access RT-PCR System) was used to generate cDNA from deaminated RNA.

The resulting cDNA was purified using the DNA Clean & Concentrator kit from Zymo and subjected to Sanger Sequencing using an ABI Prism 3730 Genetic Analyzer at the UC Davis DNA Sequencing Facility with the forward PCR primers. The sequencing peak heights were quantified in 4Peaks v1.8. Each experiment was carried out in triplicate. The editing level for the corresponding zero time point was subtracted from each data point as a background subtraction.

The results of the kinetic analysis are shown in FIG. 6 and clearly demonstrate that the cytidine analog piC, when present at the orphan base position of the AON, can enhance editing rate at the target A.

### Example 3: Kinetic ADAR Assay Comparing a Normal Cytidine (Deoxy-C; dC) and Benner's Base Z (dZ) as the Orphan Nucleotide in an RNA Editing Antisense Oligonucleotide

An identical experiment was performed using an AON carrying a Benner's base Z (dZ) as the cytidine analog opposite the target adenosine, in comparison to an AON not carrying such a cytidine analog (deoxy-C, or dC, which is different from the C in the previous example). The result of this kinetic analysis is shown in FIG. 7 and indicates that the deamination rates between piC and dZ are comparable (see also FIG. 6; both AONs with cytidine analogs display a 2-fold higher rate than an AON without a cytidine analog at that position), although the endpoint of the dZ carrying AON is somewhat lower than what was found with dC. The importance of the increased rate is that, in cells, the oligonucleotides (and ADAR) have to compete with a number of different RNA processing factors (such as splicing factors), so the faster rate may prove to be critical in ensuring that the deamination reaction can be carried out before competing factors have a chance to remove the AON and ADAR from the RNA.

### Example 4: Kinetic ADAR Assay Comparing a Normal Cytidine (Deoxy-C; dC) with Benner's Base Z (dZ) and Deoxy-Pseudoisocytidine (dpiC) as the Orphan Nucleotide in an RNA Editing Antisense Oligonucleotide

An identical experiment as above was performed using AONs carrying either a Benner's base Z (dZ) or a deoxy-pseudoisocytidine (dpiC) as the cytidine analog opposite the target adenosine, in comparison to an AON not carrying such a cytidine analog (deoxy-C; here dC). The result of this kinetic analysis is shown in FIG. 8 and shows that the endpoint is higher for both dZ and dpiC in comparison to dC. It is noted that the AONs that were synthesized for this experiment were different from the previous example. Importantly, the AON carrying a dZ as the orphan nucleotide has a faster kinetic than dC, which was also visible in FIG. 7. Also, the AON carrying dpiC as the orphan nucleotide has a faster kinetic than the AON carrying the dC as the orphan nucleotide opposite the target adenosine.

### Example 5: Determination of RNA Editing in Cells after Transfection of AONs Carrying dC and dZ as the Orphan Nucleotide

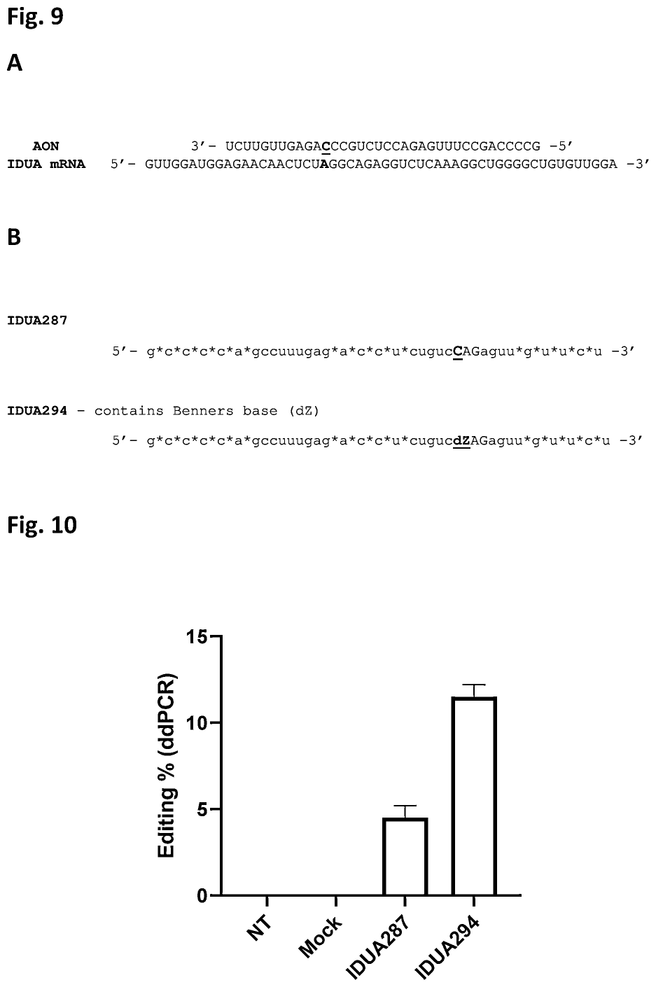

The inventors next investigated whether an AON containing the cytidine analog Benner's base Z (dZ) as the orphan nucleotide would also be more efficient in RNA editing than an identical AON carrying the deoxy-C (dC) in a cell, using endogenous ADAR. The specific editing of the same adenosine in mouse Idua mRNA, as described above, was tested. The sequence of the target and the complementary targeting AONs (somewhat longer than in FIG. 5) are given in FIG. 9.

The selected cells were primary mouse liver fibroblasts derived from a mouse strain carrying a G-to-A mutation in the Idua gene, which results in the formation of a premature stop codon (W392X). 24 h before transfection, 300,000 cells were seeded. AONs were transfected using 100 nM AON and Lipofectamine 2000 (Invitrogen) according to the manufacturer's instructions (at a ratio of 2 μl Lipofectamine 2000 to 1 μg AON). 48 h after transfection RNA was extracted using the Direct-zol RNA MiniPrep (Zymo Research) kit according to the manufacturer's instructions. cDNA was prepared using the Maxima reverse transcriptase kit 20 (Thermo Fisher) according to the manufacturer's instructions, with a combination of random hexamer and oligo-dT primers. The cDNA was diluted 3× and 1 μL of this dilution was used as template for digital droplet PCR (ddPCR) with a total input of 5 ng RNA.

The ddPCR assay for absolute quantification of nucleic acid target sequences was performed using BioRad's QX-200 Droplet Digital PCR system. 1 μl of diluted cDNA obtained from the RT cDNA synthesis reaction was used in a total mixture of 20 μl of reaction mix, including the ddPCR Supermix for Probes, dUTP (Bio Rad), and a Taqman SNP genotype assay with the following forward and reverse primers combined with the following gene-specific probes: Forward primer: 5′-CTC ACA GTC ATG GGG CTC -3′ (SEQ ID NO:8, Reverse primer: 5′-CAC TGT ATG ATT GCT GTC CAA C -3′ (SEQ ID NO:9), wild type probe (FAM NFQ labeled): 5′-AGA ACA ACT CTG GGC AGA GGT CTC A -3′ (SEQ ID NO:10), and mutant probe (HEX NFQ labeled): 5′-AGA ACA ACT CTA GGC AGA GGT CTC A -3′ (SEQ ID NO:11). 20 μl PCR mix including cDNA was filled in the middle row of a ddPCR cartridge (BioRad). Replicates were divided by two cartridges. The bottom rows were filled with 70 μl of droplet generation oil for probes (BioRad). Droplets were generated in the QX200 droplet generator. 40 μl of oil emulsion from the top row of the cartridge was transferred to a 96-wells PCR plate. The PCR 20 plate was sealed with a tin foil, and kept for 4 sec at 170° C. using the PX1 plate sealer, followed by the following PCR program: 1 cycle for 10 min at 95° C., 40 cycles for 30 sec at 95° C., 1 min at 63.8° C., 10 min at 98° C., followed by storage at 8° C. The plate was read and analyzed with a QX200 droplet reader.

The results are given in FIG. 10 and demonstrate that an AON carrying a Benner's base Z (dZ) opposite the target adenosine gives a significant higher RNA editing percentage in comparison to an identical AON that—as the only difference—carries a deoxy cytidine (dC) opposite the target adenosine.

These results show that the inventors were also capable of showing an increased efficiency of RNA editing in primary mouse fibroblasts, when the nucleotide in the AON that is directly opposite the target adenosine is a cytidine analog that serves as an H-bond donor at the N3 site.

1. An antisense oligonucleotide (AON) capable of forming a double stranded nucleic acid complex with a target RNA molecule, wherein the double stranded nucleic acid complex is capable of recruiting an ADAR enzyme for deamination of at least one target adenosine in the target RNA molecule, wherein the nucleotide in the AON that is directly opposite the at least one target adenosine is a cytidine analog that serves as an H-bond donor at the N3 site.

2. The AON according to claim 1, wherein the cytidine analog is pseudoisocytidine (piC) or Benner's base Z (dZ).

3. The AON according to claim 1 or 2, wherein the cytidine analog does not carry a 2′-OMe or a 2′-MOE ribose modification.

4. The AON according to any one of claims 1 to 3, wherein the AON comprises at least one phosphorothioate (PS), phosphonoacetate and/or methylphosphonate (MP) internucleotide linkage.

5. The AON according to any one of claims 1 to 4, wherein the AON further comprises one or more nucleotides comprising a substitution at the 2′ position of the ribose, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

6. The AON according to any one of claims 1 to 5, wherein the double stranded nucleic acid complex can recruit an endogenous ADAR enzyme, preferably an endogenous ADAR2 enzyme.

7. The AON according to any one of claims 1 to 6, wherein the AON comprises at least 15, 16, 17, 18, 19, 20, 21, 22, 23, 24, 25, 26, 27, 28, 29, 30, 31, 32, 33, 34, 35, or 36 nucleotides, and is at most 100 nucleotides long, preferably at most 60 nucleotides long.

8. A pharmaceutical composition comprising an AON according to any one of claims 1 to 7, and a pharmaceutically acceptable carrier or diluent.

9. An AON according to any of claims 1 to 7, or a pharmaceutical composition according to claim 8, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, CADASIL syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt Disease, Tay-Sachs Disease, Usher syndrome (such as Usher syndrome type I, type II, and type III), X-linked immunodeficiency, Sturge-Weber Syndrome, and cancer.

10. A method for the deamination of at least one target adenosine present in a target RNA molecule in a cell, the method comprising the steps of:
(i) providing the cell with an AON according to any one of claims 1 to 7, or a pharmaceutical composition according to claim 8;
(ii) allowing annealing of the AON to the target RNA molecule to form a double stranded nucleic acid complex capable of recruiting an endogenous ADAR enzyme in the cell;
(iii) allowing the ADAR enzyme to deaminate the target adenosine in the target RNA molecule; and
(iv) optionally identifying the presence of the deaminated adenosine in the target RNA molecule.

11. The method of claim 10, wherein step (iv) comprises:
a) sequencing a region of the target RNA molecule, wherein the region comprises the deaminated target adenosine;
b) assessing the presence of a functional, elongated, full length and/or wild type protein when the target adenosine is located in a UGA or UAG stop codon;
c) assessing the presence of a functional, elongated, full length and/or wild type protein when two target adenosines are located in a UAA stop codon;
d) assessing, when the target RNA molecule is pre-mRNA, whether splicing of the pre-mRNA was altered by the deamination; or
e) using a functional read-out, wherein the target RNA molecule after the deamination encodes a functional, full length, elongated and/or wild type protein.

12. A method for the deamination of at least one target adenosine present in a target RNA molecule, the method comprising the steps of:
(i) providing an AON according to any one of claims 1 to 7, or a pharmaceutical composition according to claim 8;
(ii) allowing annealing of the AON to the target RNA molecule to form a double stranded nucleic acid complex;
(iii) allowing a mammalian ADAR enzyme to deaminate the target adenosine in the target RNA molecule; and
(iv) optionally identifying the presence of the deaminated adenosine in the target RNA molecule.


SEQUENCE LISTING (11 sequences)

SEQ ID NO: 1 - DNA, 29 bp, Artificial sequence
  antisense oligonucleotide
  misc_feature (16)..(16): n is a, c, g, t or u
uuugagaccucugucnagaguuguucucc

SEQ ID NO: 2 - RNA, 29 nt, Mus musculus
ggagaacaacucuaggcagaggucucaaa

SEQ ID NO: 3 - DNA, 320 bp, Artificial sequence
  DNA template sequence
taatacgactcactatagggctcctcccatcctgtgggctgaacagtataacagactcccagtatacaaatggtgggagctagatattagggtaggaagccagatgctaggtatgagagagccaacagcctcagccctctgcttggcttatagatggagaacaactctaggcagaggtctcaaaggctggggctgtgttggacagcaatcatacagtgggtgtcctggccagcacccatcaccctgaaggctccgcagcggcctggagtaccacagtcctcatctacactagtgatgacacccacgcacaccccggatcc

SEQ ID NO: 4 - DNA, 28 bp, Artificial sequence
  Target FWD primer
gctcctcccatcctgtgggctgaacagt

SEQ ID NO: 5 - DNA, 25 bp, Artificial sequence
  Target RVS primer
cggggtgtgcgtgggtgtcatcact

SEQ ID NO: 6 - RNA, 54 nt, Mus musculus
guuggauggagaacaacucuaggcagaggucucaaaggcuggggcuguguugga

SEQ ID NO: 7 - DNA, 36 bp, Artificial sequence
  antisense oligonucleotide
gccccagccuuugagaccucugcccagaguuguucu

SEQ ID NO: 8 - DNA, 18 bp, Artificial sequence
  PCR primer forward
ctcacagtcatggggctc

SEQ ID NO: 9 - DNA, 22 bp, Artificial sequence
  PCR primer reverse
cactgtatgattgctgtccaac

SEQ ID NO: 10 - DNA, 25 bp, Artificial sequence
  Probe wild type
agaacaactctgggcagaggtctca

SEQ ID NO: 11 - DNA, 25 bp, Artificial sequence
  Probe mutant
agaacaactctaggcagaggtctca